(19) United States
(12) Patent Application Publication
(10) Pub. No.: US 2019/0311801 A1
(43) Pub. Date: Oct. 10, 2019

(54) DATA COLLECTION DEVICE AND METHOD FOR COLLECTING DATA FROM A MEDICAL DEVICE

(71) Applicant: Ready Care Inc., Oakville (CA)

(72) Inventors: Omar Alrayiss, Mississauga (CA); Raymond Ng, Markham (CA)

(21) Appl. No.: 16/028,566

(22) Filed: Jul. 6, 2018

(30) Foreign Application Priority Data
Apr. 6, 2018 (CA) 3000528

(51) Int. Cl. G16H 40/40; A61L 2/28

(52) U.S. Cl. CPC G16H 40/40; A61L 2/28

(57) ABSTRACT

A system for tracking the performance of a medical device including a memory, a display device; a medical device connector connected to the medical device, a processor operable to receive operational data from the medical device connector, determine a plurality of device metadata from the operational data, receive a plurality of environmental data from the medical device, determine a plurality of performance criteria based on the environmental data from the medical device, and display the performance criteria on the display device.

### FIELD

This disclosure relates to the collection of data from medical devices, such as autoclaves, more particularly for monitoring operation of the medical devices.

### BACKGROUND

The following paragraphs are not an admission that anything discussed in them is prior art or part of the knowledge of persons skilled in the art.

The benefits of sterilization of surgical instruments have long been known. One preferred method of sterilization is the use of heat and pressure in an autoclave. In an autoclave, both the temperature and pressure are increased to a predetermined threshold, and held for a predetermined amount of time.

The challenge in a modern medical setting of meeting the required standard of care for a patient during surgery requires careful attention to process and compliance. The process of sterilization including predetermined pressure, temperature and time thresholds are decided based on best practices for avoiding contamination by bacteria, viruses, fungi and spores.

Compliance on a large scale with these best practices is problematic, and the risk of injury or death to a patient due to non-compliance can be high. For instance, surgical site infections remain one of the most common causes of surgical complications, accounting for 14-17% of all hospital acquired infections (See e.g. SPAGNOLO, A. M. et al. “Operating Theatre Quality and Prevention of Surgical Site Infections.” Journal of Preventive Medicine and Hygiene 54.3 (2013): 131-137. Print.)

### SUMMARY

In a first broad aspect, there is provided a data collection device for collecting data from a medical device: a memory; a display; a medical device connector for connecting to the medical device; and a processor operable to: receive operational data from the medical device; determine a plurality of device metadata from the operational data; receive a plurality of environmental data from the medical device; determine a plurality of performance criteria based on the environmental data from the medical device; and display the performance criteria on the display.

In at least one embodiment, the data collection device may further comprise a non-volatile storage device.

In at least one embodiment, the non-volatile storage device may comprise a storage media.

In at least one embodiment, the medical device connector may be wireless.

In at least one embodiment, the medical device connector may be wired.

In at least one embodiment, the processor may be further operable to determine a medical device model from a plurality of medical device models based on the plurality of device metadata; determine a calibration model based on the medical device model; determine a plurality of calibrated performance criteria based on the plurality of performance criteria and the calibration model.

In at least one embodiment, the processor may be further operable to automatically determine the initiation of a device cycle.

In at least one embodiment, the processor may be further operable to determine a device state the operational data.

In at least one embodiment, the processor may be further operable to determine a state transition model from the medical device model.

In at least one embodiment, the processor may be further operable to record the medical device state to the non-volatile storage device.

In at least one embodiment, the processor may be further operable to create a timestamped log data file and record it to the non-volatile storage device.

In a second broad aspect, there is provided a method of collecting data from a medical device, the method comprising: receiving operational data from the medical device; determining a plurality of device metadata from the operational data; receiving a plurality of environmental data from the medical device; determining a plurality of performance criteria based on the environmental data from the medical device; and recording the plurality of device metadata and the plurality of performance criteria to a non-volatile storage device

In at least one embodiment, the method of collecting data may further comprise:

displaying each performance criteria in the plurality of performance criteria on a display device.

In at least one embodiment, the method of collecting data may further comprise: determining a medical device model from a plurality of medical device models based on the plurality of device metadata; determining a calibration model based on the medical device model; determining a plurality of calibrated performance criteria based on the plurality of performance criteria and the calibration model.

In at least one embodiment, the method of collecting data may further comprise determining automatically the initiation of a device cycle from the operational data.

In at least one embodiment, the method of collecting data may further comprise determining a device state from the operational data.

In at least one embodiment, the method of collecting data may further comprise determining a state transition model from the medical device model.

In at least one embodiment, the method of collecting data may further comprise determining if a device state transition has occurred, and if so, may record the medical device state to the non-volatile storage device.

In at least one embodiment, the recording the medical device state and recording the plurality of device metadata and the plurality of performance criteria may comprise creating a timestamped log data file and recording it to the non-volatile storage device.

In a third broad aspect, there is provided a medical device monitoring system comprising: a medical device; and a data collection device, with the medical device connector connected to the medical device.

In at least one embodiment, a medical device monitoring system may include a connection to cloud storage.

In at least one embodiment, a medical device monitoring system, wherein the medical device may comprise an autoclave.

In a fourth broad aspect, there is provided a medical device monitoring system including a computer system for receiving and analyzing data from the data collection device, and capable of identifying any data that is outside of permissible values.

### BRIEF DESCRIPTION OF THE DRAWINGS

A preferred embodiment of the present invention will now be described in detail with reference to the drawings, in which:

FIG. 1A is system diagram of components interacting with a data collection system in accordance with an embodiment of the present invention;

FIG. 1B is a hardware device diagram of the data collection system in accordance with an embodiment of the present invention;

FIG. 2 is a software component diagram of the data collection system in accordance with an embodiment of the present invention;

FIG. 3 is a method diagram of the data collection system in accordance with an embodiment of the present invention;

FIG. 4 is a method diagram of the data collection system in accordance with an embodiment of the present invention;

FIG. 5A is a state transition diagram of the data collection system in accordance with an embodiment of the present invention;

FIG. 5B is a state transition table diagram of the data collection system of FIG. 5A;

FIG. 6 is an example log file that may be produced by the data collection system in accordance with an embodiment of the present invention;

FIG. 7 is a circuit diagram of the data collection system in accordance with an embodiment of the present invention; and

FIG. 8 shows schematically another embodiment of the present disclosure.

### DESCRIPTION OF EXEMPLARY EMBODIMENTS

Various apparatuses or methods will be described below to provide an example of an embodiment of each claimed invention. No embodiment described below limits any claimed invention and any claimed invention may cover apparatuses and methods that differ from those described below. The claimed inventions are not limited to apparatuses and methods having all of the features of any one apparatus or method described below, or to features common to multiple or all of the apparatuses or methods described below. It is possible that an apparatus or method described below is not an embodiment of any claimed invention. Any invention disclosed in an apparatus or method described below that is not claimed in this document may be the subject matter of another protective instrument, for example, a continuing patent application, and the applicant(s), inventor(s) and/or owner(s) do not intend to abandon, disclaim or dedicate to the public any such invention by its disclosure in this document.

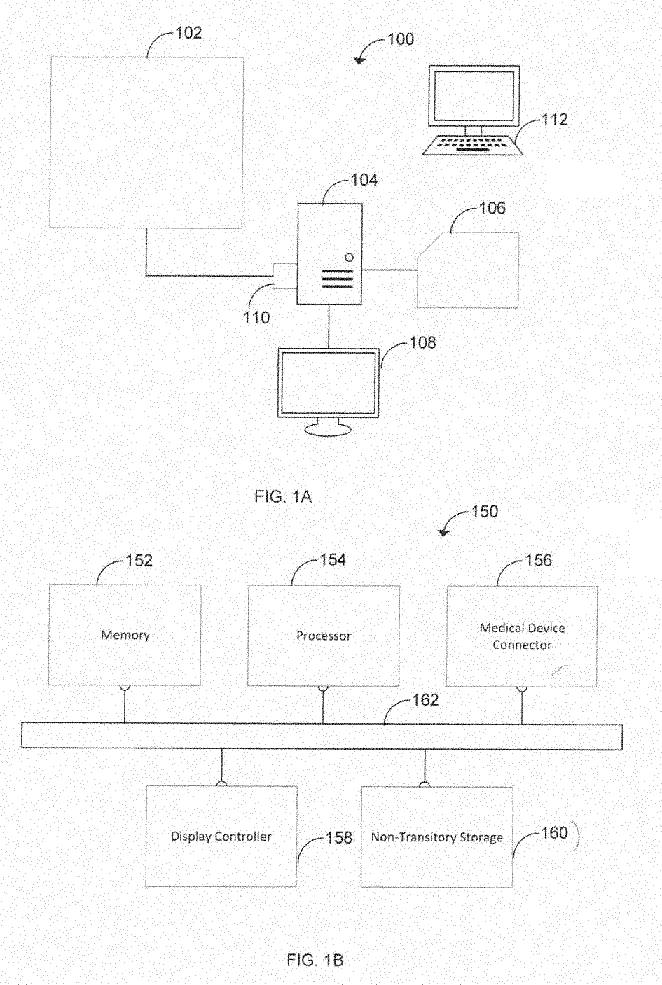

Reference is first made to FIG. 1A, which illustrates a data collection system 100. The data collection system includes a data collection device 104 connected to a non-volatile data storage media 106, which may be incorporated within the data collection device 104. The device 104 may be battery powered or connected to a conventional AC power outlet using a voltage adaptor. The data collection device 104 may be an embedded computer system such as an Arduino or an FPGA (Field Programmable Gate Array), or may be a general purpose computer system such as an Intel based personal computer, or a Raspberry Pi. The processor of the data collection device 104 may run a general purpose operating system such as Ubuntu Linux, Microsoft Windows or the like, or it may run a custom operating system.

The non-volatile data storage media 106 may include a media reader and a media card, such as a disk or a flash storage card including CompactFlash, miniSD (mini Secure Digital), microSD (micro Secure Digital) or the like. Medical device 102 may be connected to data collection device 104 using a medical device connector 110. The medical device may be an autoclave, a general control system, a cleaning device, a device that emits microwaves or x-rays, or any of the like. Log files including performance criteria about the medical device may be written to the storage media 106.

The storage media 106 may be removed from the data collection device and inserted into a media reader on a second computer device 112. The second computer device 112 may be a personal computer or a laptop. The log files on storage media 106 may then be used for follow-on operations such as attachment to email or storage on a content management system.

The medical device connector 110 may connect the medical device to the computer system using a serial connection or a parallel connection. The connection 110 between the medical device and the medical device connector may be made using an RS-232 connection, a USB (Universal Serial Bus) connection, an Ethernet connection, a Token ring connection, any sort of network based connection, or a connection custom to the medical device itself. The medical device 102 may be connected by a clocked connection or an asynchronous connection.

The medical device connector 110 may be wireless, and may communicate data with the medical device using a custom protocol or a standards based protocol such as 802.11a-i or Bluetooth.

Display 108 may be connected to data collection device 104 and content from the computer system including environment statistics or medical device state may be displayed thereupon. The display 108 may be an LCD (Liquid Crystal Display) or an LED (Light Emitting Diode) display, or the like.

Reference is first made to FIG. 1B, showing a hardware device diagram 150 of the data collection device 104 of FIG. 1A of the data collection system. FIG. 1B shows the bus 162 of data collection device 104 from FIG. 1A. The computer system has memory 152, a processor 154, a medical device connector 156, a non-transitory storage device 160, a display controller 158 and a system bus 162. The memory 152 is connected via system bus 162 and may store program instructions to be executed by processor 154. Processor 154 is connected via system bus 162 and operable to read and write to memory 152, read and write data to medical device connector 156, send data to display controller 158 for display on the display 108, and may send and receive data including log files to non-transitory storage 160. Non-transitory storage 160 may comprise the non-volatile data storage media that may be removeable, and may further comprise on chip storage or a secondary storage device used by the data collection device 104 to store program code or data.

The medical device connector 156 may include a device driver, or a plurality of device drivers operable to integrate with different types of medical devices 102. The integration of medical device connector 156 and medical device 102 may use a variety of different connection technologies, both in the physical connection of the devices and the communication protocol. The physical connection may include a serial cable or a parallel cable, a coaxial cable, a fiber-optic cable, and Ethernet cable. The communication protocol used may be custom depending on the requirements of the medical device, or may be a standard protocol such as TCP/IP (Transmission Control Protocol/Internet Protocol), RS-232 (Recommended Standard 232), IPX (Internetwork Packet eXchange), Appletalk, or the like. The medical device connector may be wireless, and may communicate data with the medical device using a custom protocol or a standards based protocol such as 802.11a-i or Bluetooth. In the case that the medical device connector is wireless, the connector may include a wireless transceiver.

The medical device connector 156 may operate to synchronize the clock signal between the medical device and the data collection device. The clock signal may represent either a clocking frequency signal. The clocking frequency signal may be in megahertz and may determine how the environmental data is recorded into buffers. The medical device connection may be made based on a symbol rate such as a baud rate, for example 9600 baud. The baud rate may represent a modulation rate in symbols per second.

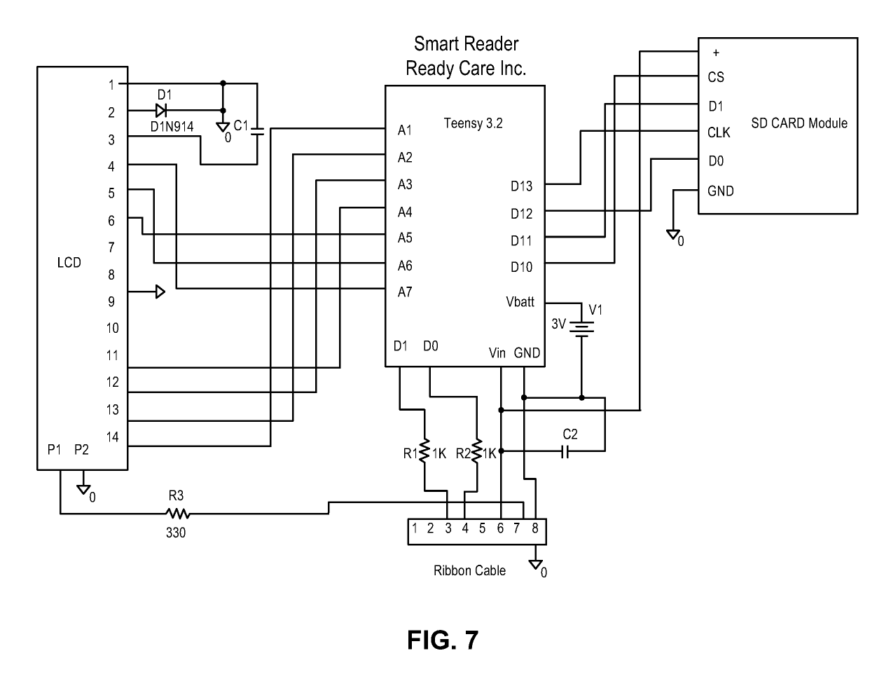

Display controller 158 may be a video card or video controller operable to encode data sent by the processor into video buffers to be shown on display 108. The display controller 158 may support different video interfaces, such as VGA (Video Graphics Array) or HDMI (High-Definition Multimedia Interface), or it may simply be connected via pinout from the data collection device 104 (see FIG. 7 for more detail).

Non-transitory storage 160 may operate to read and write data from storage media. The storage 160 may include a hard drive, a removable disk, or a memory card such as a CompactFlash, miniSD, microSD or the like. The storage 160 may function using a filesystem such as FAT (File Allocation Table), FAT32 or NTFS (New Technology File System).

System bus 162 interconnects the individual components of the computer system including memory 152, processor 154, medical device connector 156, display controller 158 and non-transitory storage 160.

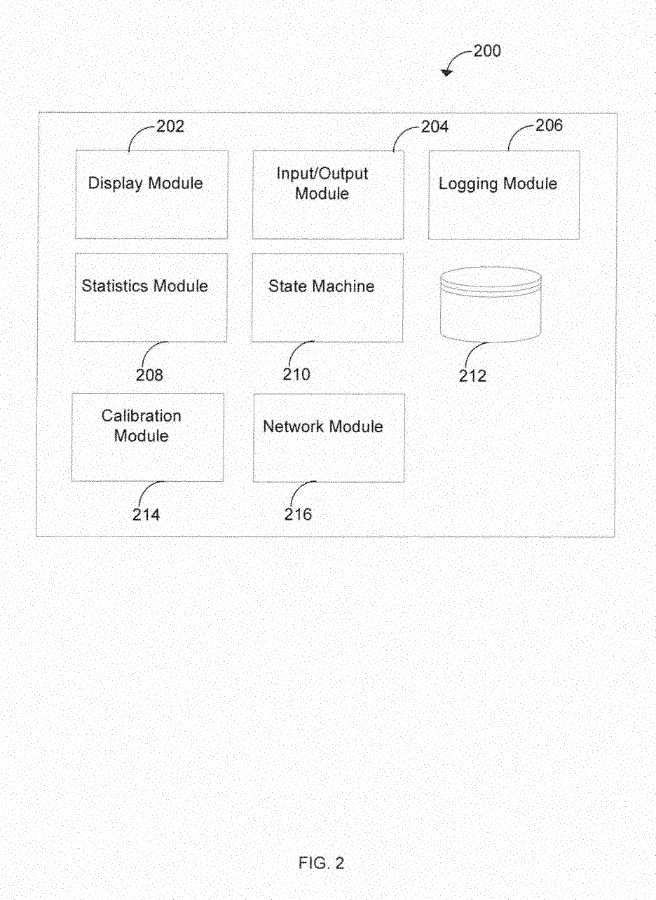

Reference is first made to FIG. 2 that illustrates a software component diagram 200 of the data collection system. The data collection system has display module 202, input output module 204, logging module 206, statistics module 208, state machine 210, database 212 and calibration module 214.

Display module 202 may be operable to provide an interface to allow the display of images or text. The interface of display module 202 may allow for program code to write to the display buffer and the written data then shown on a display, or it may display a cursor and output text on the screen at the cursor of the point. The writing to the display buffer may be in a bitmapped format or a vector based format.

Input/output module 204 may provide an interface for program code to read and write to the non-transitory storage media or read and write to the medical device using the medical device connector. The data written to the storage media using the input/output module 204 may be in the form of log files. The input/output module 204 may read operational data including device metadata from the medical device. The input/output module 204 may also receive a stream of environmental data from the medical device as it operates.

The operational data from the medical device may be in a binary format or an ASCII based format, and may be transmitted line by line, or may be clocked into a buffer. The operational data may include a model identifier, a serial identifier, a date, a time, a datetime, a cycle count, a physical device location, an attending medical professional, etc.

Logging module 206 may provide program code with an interface to log operational data, environmental data or state transitions received from the medical device connector into memory or onto the non-transitory storage media. The logging module may determine device metadata from the operational data. The device metadata may include a cycle count, a date stamp including time zone information, the device serial identifier, the device model identifier, the name of an attending physician or medical professional, or any other metadata provided from the medical device.

Statistics module 208 may provide an interface to determine statistics such as performance criteria about the medical device. The performance criteria may be determined from operational data received from the medical device via the medical device connector. The performance criteria may include average value for a time period, minimum or maximum values for a time period, device state, time of state transitions, the total elapsed time of the cycle, or any other environmental data generated by the medical device. The performance criteria may comprise values for temperature, time, pressure, volume, a displacement, a plurality of wavelengths of light, humidity, a translation, a velocity, an acceleration, a brightness, a spectroscopic measure such as from a mass spectrometer. The performance criteria may also comprise values for a biological indicator such as blood glucose levels, blood oxygenation levels, or the presence of signal molecules in a sample of blood.

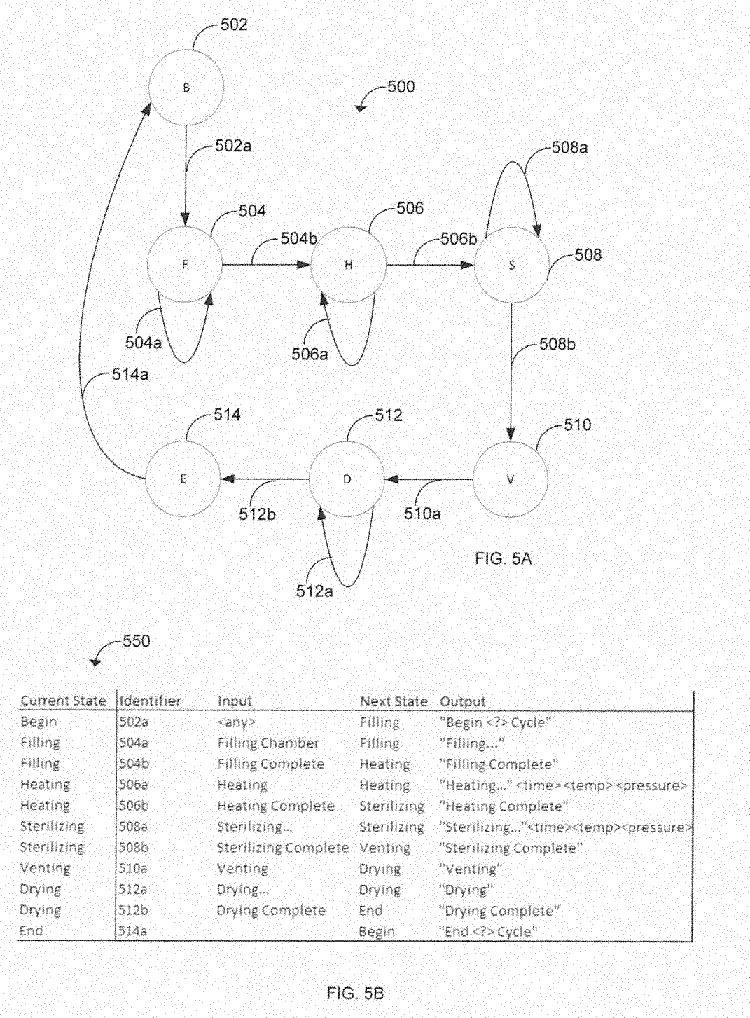

The state machine 210 may represent a set of states and state transitions for the medical device. These states and states transitions may be known prior to connection to the medical device, and may be stored in database 212, for example when the model of the medical device may be determined. The states and state transitions may be unknown prior to connection to medical device and the state machine 210 may learn the states based on the operational data received from the medical device during a cycle. The model of a medical device may be determined from a plurality of medical device models stored in the database 212 based on device metadata. The device metadata may be determined from the operational data received from the medical device. The state machine 210 may include conditions for the state transition of the medical device from one state to another. The state machine 210 may store a state transition table in database 212 that describes for each state the transitions between it and other states based on inputs from the medical device. The state machine may also be represented by a directed graph such as the one in FIG. 5. The state machine 210 may have a start state and an end state, and a transition into the end state may complete the medical device cycle, and result in the log file being completed and written to the media device. A state transition into an end state may similarly prepare for the next cycle.

Calibration module 214 may operate to allow an operator to configure calibration information for the data collection system. The calibration module 214 may also calibrate the performance criteria based on known deviations of the operational data and inaccuracies of the medical device. Such inaccuracies may include issues of range of measurement, the sensitivity of sensors, non-linearity of the sensors transfer function, deviations caused by rapid changes in measured values, noise, hysteresis, sampling frequency errors, or the like. The calibration module 214 may query the medical device for information such as a date, a time, or a datetime. This may represent a “wall clock time” that the medical device is using. The calibration module 214 may determine whether there is confidence in the reported date and time values of the medical device, and may use that date and time for calibration purposes. If the calibration module 214 determines there is not confidence in the reported date and time, a date and time tracked by the data collection device may instead be used for calibration.

The database 212 may be a relational database that operates on the computer system such as an SQL database, for example an SQLlite (Structure Query Language lite), MySQL (My Structured Query Language), or Postgres database or the like.

Similarly, the database 212 may be a non-relational database such as MongoDB. The database may instead be connected to the computer system on a network and may operate separately from the computer system of the data collection system. The database 212 may store historical data including operational data, device metadata, environmental data, state data, state transitions, performance criteria, calibration models and/or calibration data. The database 212 may also store a variety of state machine data including state transition models and calibration data for a variety of medical devices.

The Network Module 216 may provide network services to the computer system, such as TCP/IP networking. This may allow the computer system to access a database 212 that is instead located remotely.

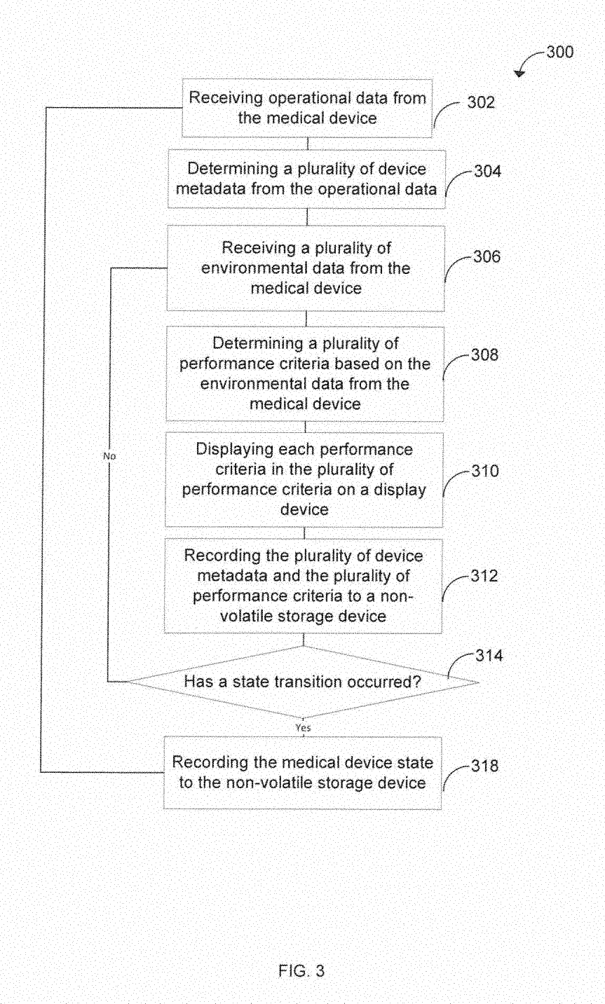

Referring to FIG. 3, where a method diagram 300 is illustrated showing one method of operation of the data collection system. At 302 the computer system receives operational data from the medical device at the medical device connector. The operational data may be received synchronously and clocked into a buffer, or may be received asynchronously.

At 304, a plurality of device metadata may be determined from the operational data by the processor of the computer system. The device metadata may include a serial number of the medical device, the model number of the medical device, or a cycle number of the device metadata. The metadata may also include information about the medical clinic or institution where it operates, or cycle metadata including the cycle count, the date, the time, etc. The device metadata may be stored in the database and may be included in the log file that is recorded to the storage media. The device metadata may be used to identify the brand and model of medical device, and this information may be used to determine device specific state machine information, state transition information, or calibration information.

At 306, a plurality of environmental data may be received from the medical device. This environmental data may or may not be calibrated, depending on the particular type of medical device. The environmental data may be stored in memory and on storage media, and may be stored in the database.

At 308, a plurality of performance criteria may be determined by the processor of the computer system. The performance criteria may be determined from the environmental data, the operational data, and the device metadata. The performance criteria may be values for temperature, time, pressure, volume, a displacement, a wavelength of light, humidity, a translation, a velocity, an acceleration, a brightness, a spectroscopic measure such as from a mass spectrometer. The performance criteria may also comprise values for a biological indicator such as blood glucose levels, blood oxygenation levels, or the presence of signal molecules in a sample of blood. The determined performance criteria may be stored in the database, stored in memory, and stored on the storage media.

At 310, the performance criteria may be displayed on the display device of the computer system. For example, a recently measure temperature may be displayed. Similarly, the state of the medical device may be displayed on the display device, for example “Heating” may be displayed.

At 312, the device metadata and the plurality of performance criteria may be recorded to the non-volatile storage media. This may involve storing the performance criteria in a database or in a log file on the storage media. Similarly, it may involve updating an existing log file previously written.

At 314, the method determines if the medical device has had state transition, and if so, records the state transition to the storage media at 318. The state transition determination 314 may be made using a state transition table in the database. The state transition table may include conditions or parameters or events that may indicate that a state transition has occurred, what the possible state transitions from a given state are, or any performance criteria that should be recorded. The state transition may also involve the determination of state level performance criteria, for instance, an average temperature, or a time taken, across the entire recorded period of the state. In the case that the state transition has not occurred, the method returns to 306 wherein it receives a plurality of environmental data.

At 318, as described above, the state transition is recorded to the storage media and the database. If the state transition moves the state machine into an End state, the recorded log file including the performance criteria and the state data may be closed, and then the state machine may be returned to a start state ready to begin again at the next medical device cycle.

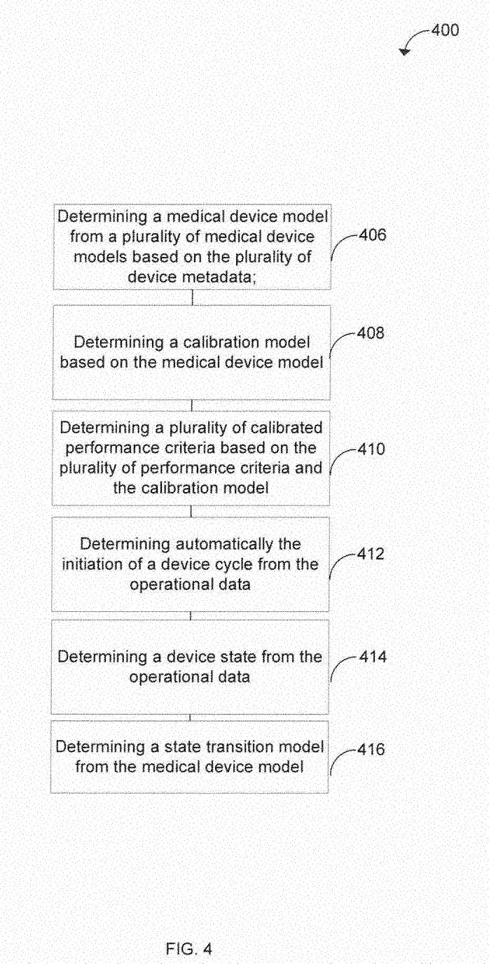

Referring to FIG. 4, a method diagram 400 of the data collection system is illustrated. At 406, having previously performed steps 302 and 304 in FIG. 3, a medical device model is determined from a plurality of medical device models based on the plurality of device metadata. The medical device model data may be stored in the database, and may include state machine information such as a state transition table relating to the operation of the medical device. The model may be determined based on a model identifier, other device metadata such as the serial identifier, or from a combination of device metadata.

At 408, having determined the medical device model, a calibration model may be retrieved from the database. The calibration model may function to calibrate the environment data points received by the data collection system. The calibration of data may be performed to correct different types of measurement errors including: issues of range of measurement, the sensitivity of sensors, non-linearity of the sensors transfer function, deviations caused by rapid changes in measured values, noise, hysteresis, sampling frequency errors, or the like.

At 410, the calibration model for the particular medical device may be used to determine a plurality of calibrated performance criteria based on the performance criteria. The calibration of performance criteria may require a fixed adjustment, a variable adjustment, filtering of data to remove noise, correlating a measured value to a known value, etc.

At 412, the data collection system may automatically determine the initiation of a device cycle based on the operational data or environmental data. The data collection system may also enter a ready or start state by an input device such as a button or a keyboard.

At 414, a device state may be determined from the operational data. The device state may be in the state transition table found in the database corresponding to the particular medical device model, or the state may be a new state. If the state is a new state, the new state may be learned by the system such that a medical device not having a state machine, state transition information, or calibration information may still interoperate.

At 416, a state transition model may be determined from the medical device model. The state transition model may be stored in the database, and may be retrievable based on a device serial identifier or device model identifier. The state transition model may be used by the state machine, and may use a state transition table in the database that describes, for each state, the transitions between it and other states based on inputs from the medical device. The state machine may also be represented by a directed graph such as the one in FIG. 5. The state machine 210 may have a start state (also known as a begin state) and an end state, and a transition into the end state may complete the medical device cycle, and result in the log file being completed and written to the media device. A state transition into an end state may similarly prepare for the next cycle.

Referring to FIG. 5A, a state transition diagram 500 is shown of the data collection system. This state transition diagram may be represented in the database by the state transition table 550 in FIG. 5B. FIG. 5A and FIG. 5B represent an example of the monitoring of an autoclave, but many different types of medical devices may be monitored and they may have different state transition diagrams that the one shown in FIG. 5A. State transitions may occur based on a predetermined threshold. In an example, the predetermined threshold may be based on temperature, time or pressure. Alternatively the medical device may trigger a state transition in the data collection system by sending a signal via the operational data or the environmental data.

State 502 reflects the Begin state of the state machine. Given an input of operational data from the medical device, the state machine follows transition 502a to Filling state 504. The operational data may inform the calibration of the environment data into calibrated performance criteria.

The state machine remains in the Filling state 504 as shown in transition 504a until the medical device signals that the chamber is filled and it is prepared for Heating state 506, at which point the state transition 504b occurs.

The state machine remains in the Heating state 506 as the medical device heats the chamber. While in the Heating state 506, the data collection system may periodically log the average temperature and pressure to the log file on the media device. The data collection system may remain in monitoring state 506 by following state transition 506a. The state transition table may similarly include a threshold for a performance criteria, for example a predetermined temperature, a predetermined pressure, or a predetermined time period that may trigger a state transition 506b. Once the predetermined threshold is met, the state machine follows state transition 506b and the state machine enters the Sterilizing state 508. Instead of a predetermined threshold, the medical device itself may signal a state transition. A state transition may be signaled by the medical device in the operational data or the environmental data.

The state machine in Sterilizing state 508 may follow state transition 508a until a predetermined threshold is met or until the medical device signals a state transition. State transition 508b occurs based on the predetermined threshold or the signal of the medical device and the data collection system enters Venting state 510. During Sterilizing state 508, the data collection system may periodically log the average temperature and pressure to the log file on the media device.

The medical device in Venting state 510 depressurizes, and the state machine of the data collection system follows transition 510a and enters Drying state 512.

The medical device remains in Drying state 512 for a predetermined period of time, or until a predetermined threshold is met, and while remaining in Drying the state machine may follow transition 512a. Once Drying 512 is complete, based upon a predetermined threshold (time, temperature, or otherwise), an event or signal from the medical device, the state machine may enter End state 514 by following state transition 512b.

Once in the End state 514, the state machine may follow transition 514a and return to the Begin state 502 in preparation for the next medical device cycle. In the End state, the data collection system may log overall performance criteria from the cycle to the log file on the media device, and may subsequently close the log file on the media device.

The log files stored on the media device may use a timestamped filename.

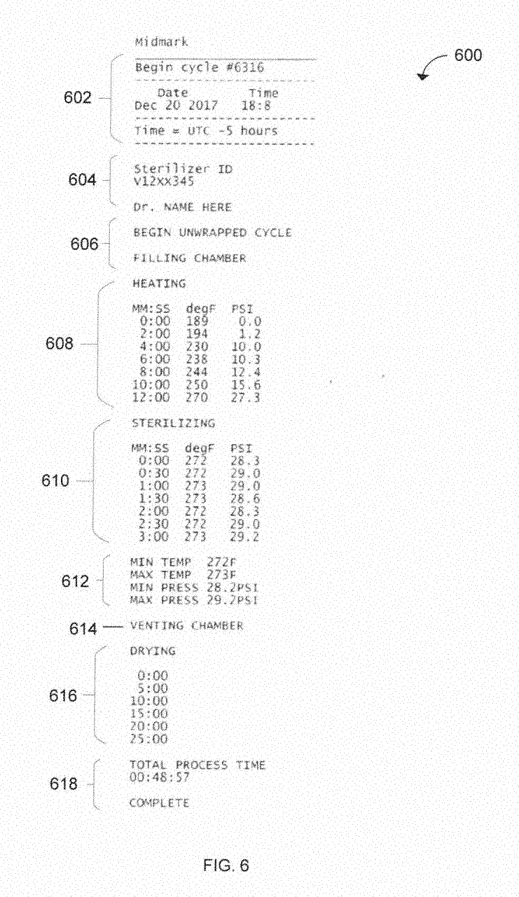

Referring to FIG. 6, an example log file 600 that may be produced by the data collection system is illustrated.

Several different types of data may be received by the data collection system. Operational data may include device specific information, such a physical parameters of the device connection, etc. The plurality of device metadata may be determined based on the operational data, for instance a model number of the medical device, the serial number of the medical device, a manufacturer of the medical device. Environmental data may include information about the execution of a cycle of a medical device, for example the internal measurements made during the execution of a cycle by an autoclave that may include instantaneous temperature and pressure readings. Performance criteria may be determined from the environmental data, and may include average values for pressure and temperature over a given time frame, maximum values or minimum values, etc.

Sections 602 and 604 may be the plurality of device metadata that is determined from the operational data. The operational data may be received from the medical device in a raw format, or the data collection system may operate to determine it automatically from a known device profile. As shown at 604, the device metadata may be determined from the operational data and this may include information about the medical device such as the brand and model of the device. In the example at 604, a medical sterilizer is shown having an identifier of V12xx345. Furthermore, information about the medical clinic or organization may be logged into the file for compliance purposes. At 604, it is shown that the name of the medical professional may be included in the log file. Other device metadata may include the cycle number, the date and time of the cycle, a medical device identifier, a physical location within a medical clinic, an attending medical professional's name, etc.

Section 606 may show the logging of the cycle including the Begin state and the Filling state.

Section 608 may show the logging of the Heating state, including logging of periodic performance criteria. Columns include time since cycle start (MM:SS), temperature (degF) and pressure (PSI). The performance criteria in this example are temperature and pressure, but there may be others. These performance criteria are logged based on a statistical average of the values over a given time period. A medical practitioner may review the heating section 608 of the log to validate whether a particular autoclave cycle reached a certain temperature and pressure and that, for example, medical implements are sufficiently sterile to be used.

At 610, the logging of the Sterilizing state is show, including logging of periodic performance criteria. Columns include time since cycle start (MM:SS), temperature (degF) and pressure (PSI). The performance criteria in this example are temperature and pressure, but there may be others. These performance criteria are logged based on a statistical average of the values over a given time period. A medical practitioner may review the sterilization section 610 of the log to validate whether a particular autoclave cycle maintained a certain temperature and pressure for the duration of that state of the cycle. In this manner the medical professional may determine that medical implements are sufficiently sterile to be used.

Sections 608 and 610 may show performance criteria determined from environmental data sent from the medical device. In the example shown in 608, for the Heating state, two minute time intervals are shown and include average temperature and pressure performance criteria. In the example shown in 610, for the Sterilizing state, two minute time intervals are shown and include average temperature and pressure performance criteria.

At 612, maximum and minimum performance criteria are also logged for the cycle, in this case temperature and pressure.

At 614, the data collection system logs a state transition into Venting state.

At 616, the data collection system logs a state transition into Drying state, showing the time-based performance criteria.

At 618, the data collection system enters the End state, and shows performance criteria across the entire cycle, in this case the length of the cycle.

Referring to FIG. 7, a circuit diagram 700 is shown of the data collection system. An arduino processor “Teensy 3.2” is shown having analog pins A1-A7 connected to an LCD display as shown, digital pins D0-D1 connected to the medical device via a ribbon cable and digital pins D10-D13 to an SD card module. The SD card module may be an IM120525008 from ITEAD and may be connected by way of a 6x1 pin header. The SD card module may include a + pin that may be connected to the Vin of the Arduino, a GND (Ground) pin that is connected to ground, a D0 pin that is connected to the D12 pin on the Arduino, a CLK (Clock) pin connected to D13 on the Arduino, a D1 pin connected to the D11 pin on the Arduino, and a CS (Chip Select) pin connected to the D10 pin on the Arduino. The SD card module may be any compatible media reader, for instance a CompactFlash, SDXC, or the like.

The processor may be of any generic type, for instance an Arduino (as shown), an FPGA (Field Programmable Gate Array), a Raspberry Pi, an Intel x86 processor, an AMD (Advanced Micro Devices) processor, or an ARM (Advanced RISC Machine) processor.

The LCD display may be a NHD-C0220AZ-FSW-FTW from Newhaven Display Intl. Pins 1, 2, and 3 are configured to provide a contrast control for the LCD. Pin P1 is attached to pin 7 on the ribbon cable. Pin P2 is attached to ground. Pin 4 on the LCD controls the register select and is attached to analog pin A7 on the Arduino. Pin 5 on the LCD controls read/write select, and is attached to pin A6 on the Arduino. Pin 11-14 control four high order bidirectional three-state data bus lines that are connected to pins A4-A1 respectively.

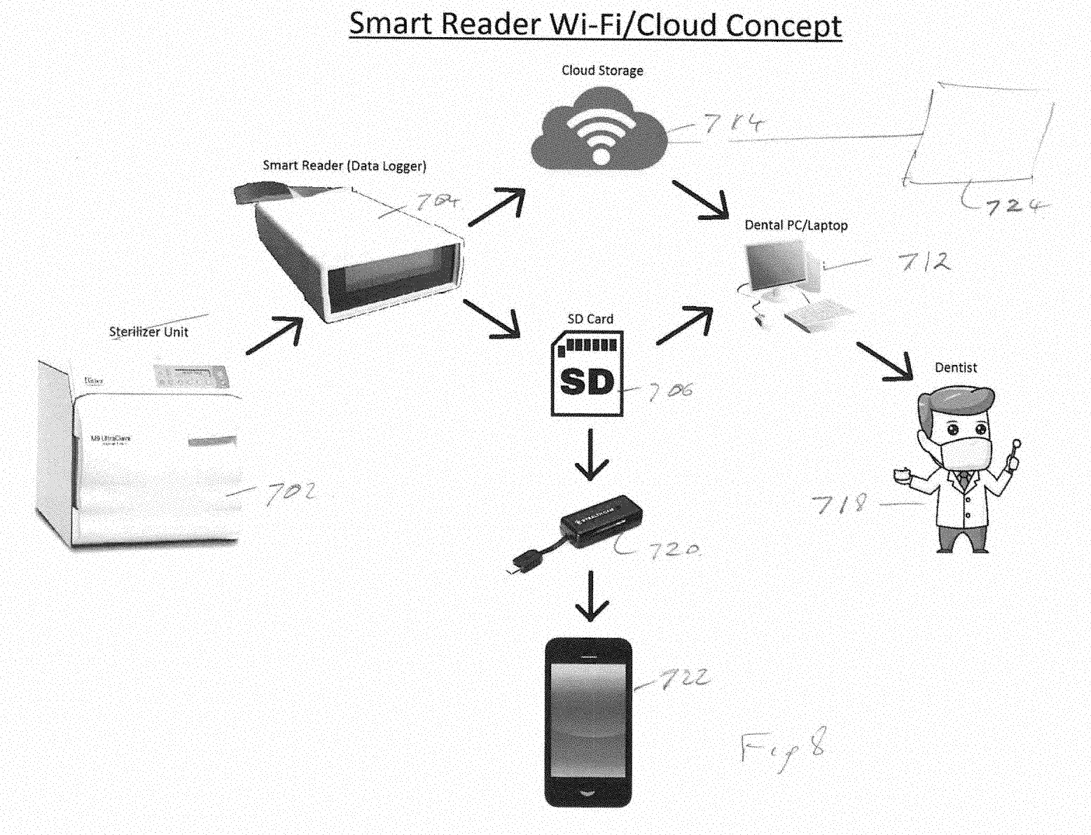

Reference will now be made to FIG. 8, which shows schematically another embodiment of the present disclosure. For consistency, like components will be identified with the reference 7XX corresponding to the references 1XX in FIG. 1A.

As previously described, a medical device, for example a sterilizer unit, 702 may be connected to a data collection device 704. A non-volatile data storage media 706 may be mounted in or connected to the data collection device 704, for recording and storing data collected. The data storage media 706 may also be connected to a second computer device 712, which may, for example, be a computing device or part of computing facilities at a dental or medical office.

Further, the data collection device 704 is connected to cloud storage indicated at 714, and this cloud storage 714 may be accessible by the second computing device 712. Such cloud storage may eliminate the requirement to provide the data storage media 706.

As further shown, a media reader 720 can be provided that is connectable to a smart phone 722. The data storage media 706, for example an SD storage card may then be inserted into the media reader 720, so that the data stored thereon can be read and displayed on the smart phone 722.

The cloud storage 714 may also be accessible by a regulatory body, for example a government regulator or the like, indicated schematically at 724. The regulatory body 724 may also have communication via the cloud storage 714 or otherwise to the computing device 712. The regulatory body can then, for example at periodic intervals, access data stored in the cloud storage 714, and check that appropriate regulatory requirements are being met, e.g. to ensure that proper sterilization protocols are being followed. The regulatory body 724 can then report to the computing device 712, effectively reporting to the person or persons (indicated at 718) responsible for operating the sterilizer or other medical device 702, with a report indicating whether or not sterilization protocols and procedures are in compliance with regulatory requirements.

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

Various embodiments have been described herein by way of example only. Various modification and variations may be made to these example embodiments without departing from the spirit and scope of the invention, which is limited only by the appended claims. Also, in the various user interfaces illustrated in the figures, it will be understood that the illustrated user interface text and controls are provided as examples only and are not meant to be limiting. Other suitable user interface elements may be possible. In the claims, it will be understood that the individual elements and method steps may be combined together in any practical combination. More particularly and without limitation, the medical device monitoring system may include any of the defined or claimed data collection devices.

We claim:

1. A data collection device for collecting data from a medical device:
a memory;
a display;
a medical device connector for connecting to the medical device; and
a processor operable to:
receive a plurality of operational data from the medical device;
determine a plurality of device metadata from the operational data;
receive a plurality of environmental data from the medical device;
and
at least one of display data on the display and store the data.

2. The data collection device of claim 1, wherein the processor is operable to determine a plurality of performance criteria based on the environmental data from the medical device, and operable to display the plurality of performance criteria on the display.

3. The data collection device of claim 1, further comprising a non-volatile storage device for storing data.

4. The data collection device of claim 3, wherein the non-volatile storage device comprises a storage media.

5. The data collection device of claim 4, wherein the medical device connector is wireless.

6. The data collection device of claim 4, wherein the medical device connector is wired.

7. The data collection device of claim 1, wherein the processor is further operable to
determine a medical device model from a plurality of medical device models based on the plurality of device metadata;
determine a calibration model based on the medical device model;
determine a plurality of calibrated performance criteria based on the plurality of performance criteria and the calibration model.

8. The data collection device of claim 7, wherein the processor is further operable to automatically determine the initiation of a device cycle.

9. The data collection device of claim 8, wherein the processor is further operable to determine a device state the operational data.

10. The data collection device of claim 9, wherein the processor is further operable to determine a state transition model from the medical device model.

11. The data collection device of claim 10, wherein the processor is further operable to record the medical device state to the non-volatile storage device.

12. The data collection device of claim 11, wherein the processor is further operable to create a timestamped log data file and record it to the non-volatile storage device.

13. A method of collecting data from a medical device, the method comprising:
receiving a plurality of operational data from the medical device;
determining a plurality of device metadata from the operational data;
receiving a plurality of environmental data from the medical device; and
at least one of: recording data on a non-volatile storage device; and transmitting data for storage and analysis.

14. The method of claim 13, wherein the method includes: determining a plurality of performance criteria based on the environmental data from the medical device; and at least one of recording on the non-volatile storage device and transmitting, the plurality device metadata and the plurality of performance criteria.

15. The method of claim 14, further comprising:
displaying each performance criteria in the plurality of performance criteria on a display device.

16. The method of claim 13, further comprising:
determining a medical device model from a plurality of medical device models based on the plurality of device metadata;
determining a calibration model based on the medical device model;
determining a plurality of calibrated performance criteria based on the plurality of performance criteria and the calibration model.

17. The method of claim 16, further comprising
determining automatically the initiation of a device cycle from the operational data.

18. The method of claim 17, further comprising determining a device state from the operational data.

19. The method of claim 18, further comprising determining a state transition model from the medical device model.

20. The method of claim 19, further comprising determining if a device state transition has occurred, and if so, at least one of: recording the medical device state to the non-volatile storage device; and transmitting the medical devise state.

21. The method of claim 20, wherein each of the recording and transmitting: the medical device state; recording the plurality of device metadata; and the plurality of performance criteria comprises creating a timestamped log data file and, respectively, recording the data to the non-volatile storage device and transmitting the data.

22. The method of claim 13, the method including transmitting data for storage at a remote storage facility.

23. The method of claim 22, including: providing a regulatory body with access to the data at the remote storage facility; permitting the regulatory body to check that performance criteria are within permitted values; and enabling the regulatory body to communicate with an entity responsible for the medical device to report compliance and non-compliance of the performance criteria with the permitted values.

24. The method of claim 22, including providing access to the data stored at the remote storage facility by an entity responsible for the medical device.

25. The method of claim 13, wherein the medical device comprises an autoclave in a dentist's office, and an entity responsible for the medical device is an entity responsible for the dentist's office.

26. A medical device monitoring system comprising:
A medical device; and
a data collection device as claimed in claim 1, with the medical device connector connected to the medical device.

27. The medical device monitoring system as claimed in claim 26, including a connection to cloud storage.

28. The medical device monitoring system as claimed in claim 26, wherein the medical device comprises an autoclave.

29. The medical device monitoring system as claimed in any one of claim 26, including a computer system for receiving and analyzing data from the data collection device, and capable of identifying any data that is outside of permissible values.
